(19) United States
(12) Patent Application Publication
(10) Pub. No.: US 2011/0184300 A1
(43) Pub. Date: Jul. 28, 2011

(54) ATRIAL FIBRILLATION DETECTION BASED ON ABSENCE OF CONSISTENT P-LOOPS IN AVERAGED VECTORCARDIOGRAM

(75) Inventors: Alexei Shvilkin, Chestnut hill, MA (US); Bosko Bojovic, Belgrade (RS); Ljupco Hadzievski, Belgrade (RS); Dorin Panescu, San Jose, CA (US)

(73) Assignee: NewCardio, Inc., Sant Clara, CA (US)

(21) Appl. No.: 12/694,236

(22) Filed: Jan. 26, 2010

(51) Int. Cl. A61B 5/046

(52) U.S. Cl. 600/512

(57) ABSTRACT

A method for determining the presence of atrial fibrillation includes obtaining data from at least three orthogonal leads indicative of electrical activity of a heart over multiple heart beats, averaging the data to obtain an average heart beat, generating, from the averaged data, an average heart beat vectorcardiogram (VCG) that is a function of voltage level, comparing, over a segment of the average heart beat, the average heart beat VCG with a threshold value, and indicating whether the average heart beat VCG exceeds or does not exceed the threshold value.

### TECHNICAL FIELD

The field of this disclosure is medical diagnostics, and more particularly, detection of atrial fibrillation (Afib) based on measurement and recording of bioelectric signals, using tools such as electrocardiograms (ECGs) and vectorcardiograms (VCGs).

### BACKGROUND

Atrial fibrillation (Afib) is the most common, abnormal rhythm of the heart. Normally, the heart contracts and pumps blood with a regular rhythm. This rhythm is triggered by regular electrical discharges that travel through the heart and cause the muscle of the heart to contract. The electrical discharges, originating in the sino-atrail (SA) node and subsequently echoed in the atrio-ventricular (AV) node, trigger contractions in the corresponding heart tissue and result in the pumping action of the heart. In a normal heart, the rate of atrial contraction is the same as the rate of ventricular contraction.

During Afib, electrical discharges are not generated solely by the SA node. Instead, electrical discharges are sourced by other parts of the atria. These abnormal discharges are rapid and irregular and may exceed 350 discharges per minute. The rapid and irregular discharges cause ineffective contractions of the atria, which quiver rather than beat as a unit, reducing their ability to pump blood into the ventricles.

The rapid and irregular electrical discharges from the atria in Afib then pass through the AV node and into the ventricles, causing the ventricles to contract irregularly and rapidly. The contractions of the ventricles may average 150/minute, much slower than the rate in the atria, as they are unable to contract at 350/minute rate of the atria. Even at this lower average rate of 150/minute, the ventricles may not have enough time to fill maximally with blood before the next contraction, particularly without the normal contraction of the atria. Thus, Afib decreases the amount of blood pumped by the ventricles because of their rapid rate of contraction and the absence of normal atrial contractions.

Afib is a common disorder, with half a million new cases diagnosed annually in the United States, and billions of dollars spent on diagnosis and treatment. Two related diagnostic tools available to medical professionals are the electrocardiogram (ECG) and the vectorcardiogram (VCG). A standard 12-lead ECG is a graphical representation of the electrical activity of the heart, showing different waves that represent the sequence of depolarization and repolarization of the atria and ventricles. The 12-lead ECG is obtained using a plurality of electrodes (ten typically) disposed at various standard locations on the patient's body, and electrical activity thereby obtained is graphically displayed for interpretation and diagnosis. The graphical display can be on paper, on which the ECG is recorded at a speed of 25 mm/sec, and the voltages are calibrated so that 1 mV=10 mm in the vertical direction. FIG. 1 shows an example 12-lead ECG. A VCG by comparison is a tracing of the direction and magnitude of the heart's electrical activity, as derived from the same or similar electrode placement as the ECG, and as viewed from different planes relative to the heart. The VCG is a vector representation produced by an oscilloscope simultaneously recording three standard leads. FIG. 2 shows an example VCG, providing views in three planes: frontal, saggital and horizontal.

### OVERVIEW

Described herein is a method for determining the presence of atrial fibrillation. The method includes obtaining data from at least three orthogonal leads indicative of electrical activity of a heart over multiple heart beats, averaging the data to obtain an average heart beat, generating, from the averaged data, an average heart beat vectorcardiogram (VCG) that is a function of voltage level, comparing, over a segment of the average heart beat, the average heart beat VCG with a threshold value, and indicating whether the average heart beat VCG exceeds or does not exceed the threshold value.

Also described herein is a program storage device readable by machine, embodying a program of instructions executable by the machine to perform a method. The method includes obtaining data from at least three orthogonal leads indicative of electrical activity of a heart over multiple heart beats, averaging the data to obtain an average heart beat, generating, from the averaged data, an average heart beat vectorcardiogram (VCG) that is a function of voltage level, comparing, over a segment of the average heart beat, the average heart beat VCG with a threshold value, and indicating whether average heart beat VCG exceeds or does not exceed the threshold value.

Also described herein is a system for determining the presence of atrial fibrillation. The system includes a computer configured to receive data from at least three orthogonal leads indicative of electrical activity of a heart over multiple heart beats. The computer is also configured to average the data to obtain an average heart beat, generate, from the averaged data, an average heart beat vectorcardiogram (VCG) that is a function of voltage level, compare, over a segment of the average heart beat, the average heart beat VCG with a threshold value, and identify the average heart beat VCG as either exceeding or not exceeding the threshold value. The system also includes an indicator for providing an indication of the identification.

Also as described herein, a method for determining the presence of atrial fibrillation includes obtaining data from at least three orthogonal leads indicative of electrical activity of a heart over multiple heart beats, performing ensemble processing to generate a generalized heart beat, generating a generalized vectorcardiogram (VCG) that is a function of voltage level, comparing, over a segment of the generalized heart beat, the generalized VCG with a threshold value, and indicating whether the generalized VCG exceeds or does not exceed the threshold value.

Also as described herein, a system for determining the presence of atrial fibrillation includes a computer configured to receive data from at least three orthogonal leads indicative of electrical activity of a heart over multiple heart beats, and an indicator configured to provide an indication of an identification performed by the computer. Computer is configured to perform ensemble processing to generate a generalized heart beat, generate a generalized vectorcardiogram (VCG) that is a function of voltage level, compare, over a segment of the generalized heart beat, the generalized VCG with a threshold value, and identify the generalized VCG as either exceeding or not exceeding the threshold value.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

In the drawings:

FIG. 1 is an example 12-lead ECG;

FIG. 2 is an example VCG, providing views in three planes: frontal, saggital and horizontal;

FIG. 3 is a flow diagram of a method for constructing an average heart beat VCG;

FIG. 4 is schematic diagram of a standard 12-lead ECG, depicting the activity of three of leads, I, II and III, extending;

FIG. 4A illustrates selection of the fiducial point R from the standard ECG of FIG. 4;

FIG. 4B is a schematic illustration of the superimposition of beats for each of three leads;

FIG. 4C is a schematic illustration of an average heart beat for each of three leads;

FIGS. 5A-5C are depictions of average heart beat VCG in 3-D in the absence of Afib;

FIG. 5D shows the segment over the depiction in FIGS. 5A-5C is taken;

FIGS. 5D-5G are depictions of average heart beat VCG in 3-D in the presence of Afib;

FIG. 5H shows the segment over the depiction in FIGS. 5D-5G is taken;

FIG. 6 is a flow diagram of an analysis method for indicating the presence or absence of Afib;

FIG. 7 is a schematic diagram of a system for implementing Afib indication.

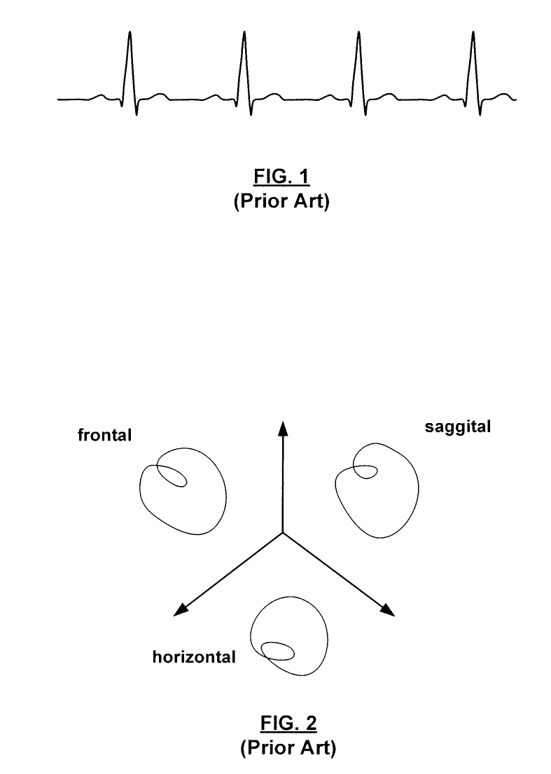

### DESCRIPTION OF EXAMPLE EMBODIMENTS

Example embodiments are described herein in the context of atrial fibrillation detection based on absence of consistent P-loops in an averaged vectorcardiogram. Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used to the extent possible throughout the drawings and the following description to refer to the same or like items.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with this disclosure, the components, process steps, and/or data structures described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Eraseable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card, paper tape and the like) and other types of program memory.

A method and system are described herein for atrial fibrillation (Afib) detection based on absence of consistent p-loops in an averaged vectorcardiogram. Such detection is facilitated by the fact that in an averaged vectorcardiogram, the absence of consistent P-loops is indicative of Afib. Conversely, the presence of consistent P-loops is indicative of sinus rhythm and/or atrial flutter.

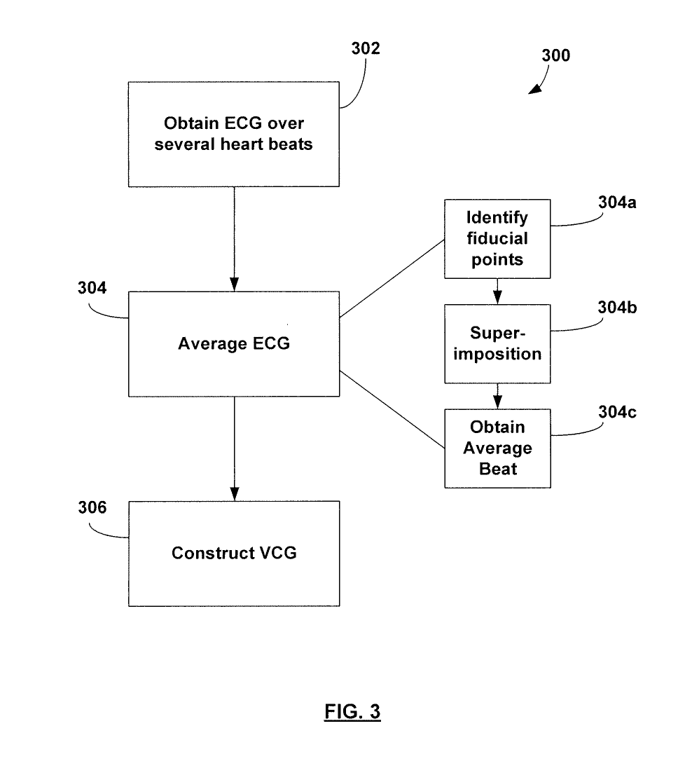

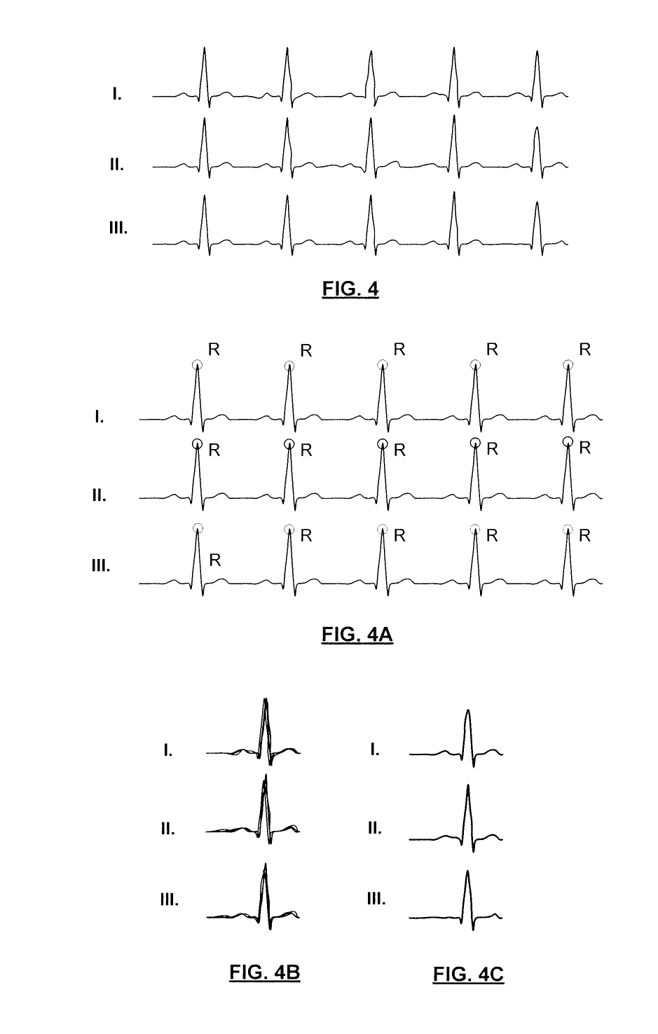

With reference to FIG. 3, a flow diagram of a method 300 for constructing an average heart beat VCG is performed. It will be appreciated that averaging as used herein is only one, specific example of ensemble processing that is generally applicable to achieve the desired generalized VCG for further analysis. Other specific examples of applicable ensemble processing techniques include, but are not limited to, median filtering and median beats. Method 300 includes obtaining, at 302, data representative of the electrical activity (depolarization and repolarization) of the heart over multiple heart beats, preferably over five to ten or more beats. A standard 12-lead ECG, either using a conventional 10-electrode system or using systems or devices having a reduced set of electrodes, such as eight, or such as the reduced number in the device described in U.S. Pat. No. 7,647,093 (Bojovic et al.), the contents of which are incorporated herein in their entirety, can serve this purpose. As one example, it is possible to use a mere three orthogonal leads to generate the necessary electrical activity data. The foregoing description, however, will be in terms of the standard 12-lead ECG, with FIG. 4 schematically depicting activity of three of these leads, I, II and III, extending over a set of five heart beats.

Referring again to FIG. 3, after the ECG is obtained, an averaging operation is performed, at 304. The averaging operation includes several steps, delineated 304a-304f. In step 304a, R wave peaks, or similar commonly-identifiable points, are identified for each of the 12 leads, as depicted in FIG. 4A. Identification can be manual or automatic, in the latter case using for instance commercial software such as the QTinno™ software suite available from NewCardio of Santa Clara, Calif. The identified R wave peaks are used as the fiducial points in the ensuing analysis.

At step 304b, a superimposition is performed, wherein, for each lead, all beats are superimposed over each other, with their fiducial R points (R wave peaks) being aligned. This is depicted in FIG. 4B. Then, from this superimposition, an average beat is obtained for each lead, at 304c. The averaged beat is shown in FIG. 4C. As explained above, averaging operation 304 is a specific example of a portion of a more general ensemble processing operation that is performed, and, in the general case, would result in a generalized heart beat. In the specific averaging example, the generalized heart beat is the averaged beat.

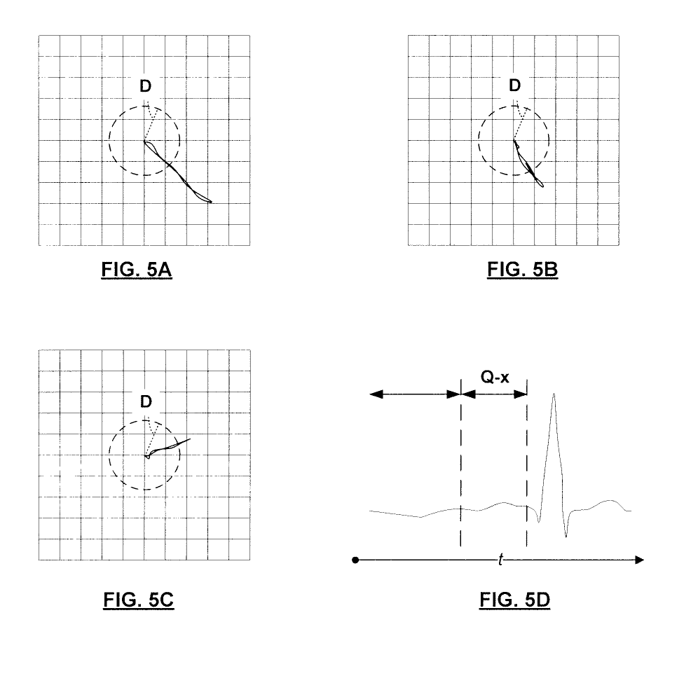

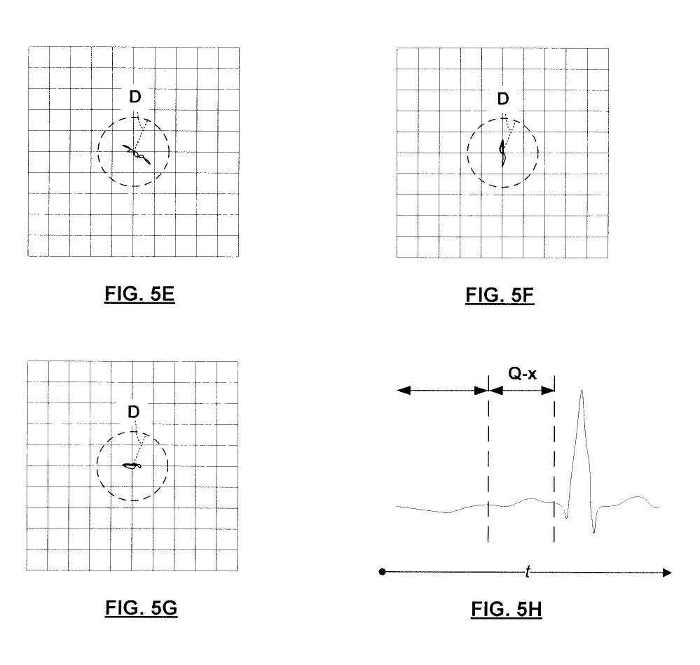

After completion of the averaging operation 304, an average vectorcardiogram is constructed, using any known method, such as a Dower matrix. The construction operation is delineated 306 in FIG. 3. Two examples of such a constructed average VCG are shown in 3-D views in FIGS. 5A-5C and 5E-5G. While described herein as using a VCG, it will be appreciated that this is for convenience only. In fact the data and analysis need not result in or rely on any graphical representation of the derived data, particularly in a fully automated application.

It should be recognized that the order of the aforementioned steps and operations is not limited to that described above. For instance, the average VCG can be derived by first constructing VCGs for each of the processed beats, and then averaging the individual beat VCGs together. The individual beat VCGs can be aligned in the 3-D space by using the same reference point and the R point, which in that case would correspond to the tip of the QRS loop.

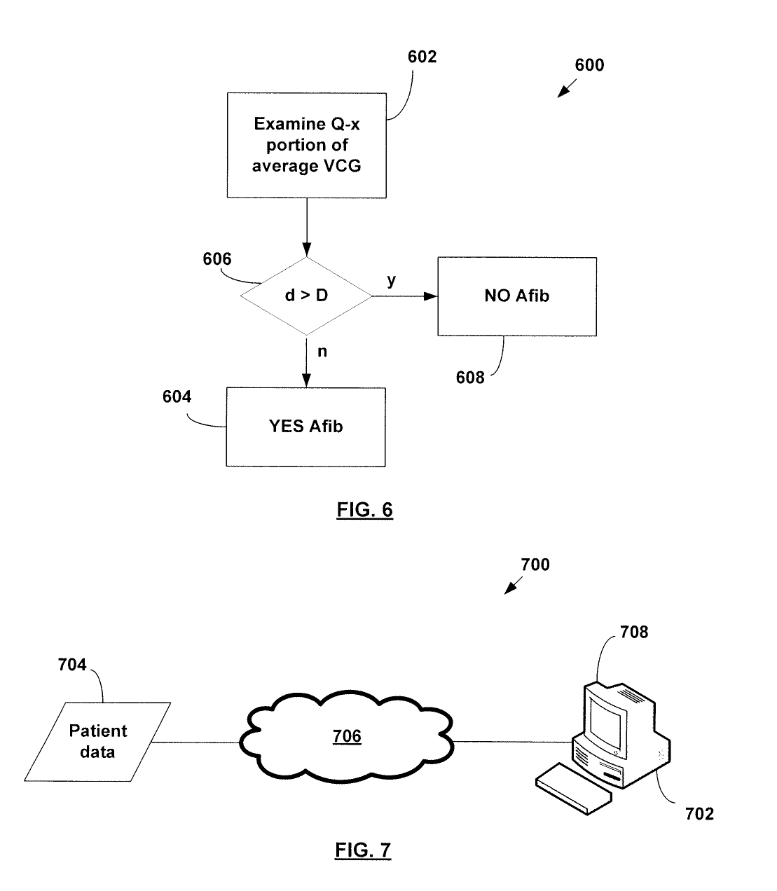

The averaging method 300 is followed by an analysis method 600, described with reference to FIG. 6. At 602, the portion of the average VCG beginning from a selected upstream point Q-x (where x is either milliseconds or percent of the RR interval—for example, x could be in the range of about 5 to about 50 ms to the left of the Q point, or in the range of about 0.5 to about 10% of the RR interval) on the ECG (FIGS. 5D and 5H), all the way to a second selected pout further upstream, for example at the beginning of the cardiac cycle, is examined. “Upstream” in this sense designates earlier in time. Alternatively, the examined portion could extend left (upstream) of Q-x to the end of the preceding T wave. In particular, Afib is declared, at 604, if the condition at 606 is met—that is, if there is no 3-D curve with a diameter d, defined as the longest distance between two points on the curve, that is greater than a preset amount D. This is the case in FIGS. 5E-5G, evidencing Afib. Conversely, if there is 3-D curve with a diameter d greater than the present amount D, then no Afib is declared, at 608. This is the case in FIGS. 5A-5C. It will be appreciated that d and D actually represent electrical voltage levels signifying heart electrical activity. Example values for the threshold D is the range from about 0.1 mV to about 0.3 mV. A specific value for D can be about 0.15 mV. D can be adjustable by the operator or physician depending on the patient or population of patients being examined. It can also be represented differently, depending on preference and the particular application. For instance, it can be depicted on a millimetric scale, or one labeled in millivolts, and so on.

It will be appreciated that the above determinations can be performed visually/manually by a physician for example, and/or automatically by a suitably programmed processor. Alternatively, some tasks may be performed visually/manually and others automatically, depending on the design choice, urgency, or other constraints.

As can be concluded from the above discussion, in Afib, averaging removes the fibrillatory waves (F-waves) because they are asynchronous with respect to the R fiducial points. By contrast, in sinus rhythm or atrial flutter (a known confounding rhythm in such diagnosis), the waves residing to the left of the R fiducial point are synchronous to the R point. As such, averaging does not affect their morphology. When presented in a 3-D VCG as shown above, the effects of these characteristics become clearly visible because the averaged F-waves amount to a small 3-D “cluster” that reflects the baseline noise, whereas in sinus rhythm or atrial flutter, the same waves develop into visible loops. In sinus rhythm, there is just one P loop corresponding to the P wave. In atrial flutter, there is a plurality of loops because, typically, atrial flutter presents with a constant ratio of the atrial heart rate to the ventricular heart rate.

As explained above, some or all of the operations involved can be conducted using a suitably programmed processor. A more complete example of a system is shown in FIG. 7. System 700 includes a computer 702 to which patient data 704 is sent, either in real-time or through a storage medium (not shown). The data may originate from a remote location and be transmitted over a network 706. Computer 702 includes a processor (not shown) programmed with executable instructions for carrying out some or all of the above methods, and/or for displaying the patient data for analysis by a caretaker. Display 708, which can be a CRT or a flat-screen type monitor or a projector or the like, is provided for this purpose, and can be used to display the average heart beat VCG, or to simply indicate whether or not the threshold value D is exceeded, so that further, manual analysis can be conducted if necessary. Of course in the case of a basic identification, a display would not be necessary, as any known identification can be used, including those perceptible by a human such as a simple LED or speaker or horn or the like. It may also possible to selectively display (or indicate) the average heart beat VCG depending on whether the threshold D, or a related threshold D′ that can be greater or less than D, is exceeded. In this manner, a more conservative threshold can be set for automatic determination by the computer 702, so that candidate Afib cases can be presented for further scrutiny by a physician, who can then apply a more exacting threshold D. Computer 702 can also transmit information relating to the analysis or acquired data to other devices and locations, through network 704 or other networks or pathways that may be wired or wireless.

While embodiments and applications have been shown and described, it would be apparent to those skilled in the art having the benefit of this disclosure that many more modifications than mentioned above are possible without departing from the inventive concepts disclosed herein. The invention, therefore, is not to be restricted except in the spirit of the appended claims.

What is claimed is:

1. A method for determining the presence of atrial fibrillation, comprising:
obtaining data from at least three orthogonal leads indicative of electrical activity of a heart over multiple heart beats;
performing ensemble processing to generate a generalized heart beat;
generating a generalized vectorcardiogram (VCG) that is a function of voltage level;
comparing, over a segment of the generalized heart beat, the generalized VCG with a threshold value; and
indicating whether the generalized VCG exceeds or does not exceed the threshold value.

2. The method of claim 1, further comprising declaring atrial fibrillation if the threshold value is not exceeded.

3. The method of claim 1, further comprising declaring no atrial fibrillation if the threshold value is exceeded.

4. The method of claim 1, wherein obtaining data from at least three orthogonal leads comprises obtaining data from a standard 12-lead ECG (electrocardiogram).

5. The method of claim 1, wherein the segment spans from a first selected point upstream of a Q point of the generalized heart beat to a second selected point further upstream therefrom.

6. The method of claim 5, wherein the first selected point is about 5 to about 50 milliseconds downstream of the Q point.

7. The method of claim 5, wherein the first selected point is about 0.5 to about 10% of an R-R interval of the generalized heart beat.

8. The method of claim 5, wherein the second selected point is at the beginning of a cardiac cycle.

9. The method of claim 5, wherein the second selected point is at the end of a preceding T wave.

10. The method of claim 1, wherein generating a generalized heart beat comprises superimposing multiple heart beats using a common fiducial point.

11. The method of claim 11, wherein the common fiducial point is the R point in the cardiac cycle.

12. The method of claim 1, wherein the generalized VCG is generated from the generalized heart beat.

13. The method of claim 1, wherein the generalized VCG is generated from a plurality of VCGs each corresponding to one of the multiple heart beats.

14. The method of claim 1, wherein the threshold is in the range of about 0.1 millivolts to about 0.3 millivolts.

15. The method of claim 1, wherein the threshold is about 0.15 millivolts.

16. The method of claim 1, further comprising selectively displaying the generalized VCG based on whether or not the threshold is exceeded.

17. The method of claim 1, wherein the ensemble processing comprises median filtering.

18. The method of claim 1, wherein the ensemble processing comprises generating a median beat.

19. A system for determining the presence of atrial fibrillation, comprising:
a computer configured to receive data from at least three orthogonal leads indicative of electrical activity of a heart over multiple heart beats, the computer further configured to:
perform ensemble processing to generate a generalized heart beat;
generate a generalized vectorcardiogram (VCG) that is a function of voltage level;
compare, over a segment of the generalized heart beat, the generalized VCG with a threshold value; and
identify the generalized VCG as either exceeding or not exceeding the threshold value; and
an indicator configured to provide an indication of the identification.

20. The system of claim 19, wherein the computer is further configured to declare atrial fibrillation if the threshold value is not exceeded.

21. The system of claim 19, wherein the computer is further configured to declare no atrial fibrillation if the threshold value is exceeded.

22. The system of claim 19, wherein obtaining data from at least three orthogonal leads comprises obtaining data from a standard 12-lead ECG (electrocardiogram).

23. The system of claim 19, wherein the segment spans from a first selected point upstream of a Q point of the generalized heart beat to a second selected point further upstream therefrom.

24. The system of claim 23, wherein the first selected point is about 5 to about 50 milliseconds downstream of the Q point.

25. The system of claim 23, wherein the first selected point is about 0.5 to about 10% of an R-R interval.

26. The system of claim 23, wherein the second selected point is at the beginning of a cardiac cycle.

27. The system of claim 23, wherein the second selected point is at the end of a preceding T wave.

28. The system of claim 19, wherein generating a generalized heart beat comprises superimposing multiple heart beats using a common fiducial point.

29. The system of claim 28, wherein the common fiducial point is the R point in the cardiac cycle.

30. The system of claim 19, wherein the generalized VCG is generated from the generalized heart beat.

31. The system of claim 19, wherein the generalized VCG is generated from a plurality of VCGs each corresponding to one of the multiple heart beats.

32. The system of claim 19, wherein the threshold is in the range of about 0.1 millivolts to about 0.3 millivolts.

33. The system of claim 19, wherein the threshold is about 0.15 millivolts.

34. The system of claim 19, further comprising a display for selectively displaying the generalized VCG based on whether or not the threshold is exceeded.

35. The system of claim 19, wherein the ensemble processing comprises median filtering.

36. The system of claim 19, wherein the ensemble processing comprises generating a median beat.

37. A method for determining the presence of atrial fibrillation, comprising:
obtaining data from at least three orthogonal leads indicative of electrical activity of a heart over multiple heart beats;
averaging the data to obtain an average heart beat;
generating, from the averaged data, an average heart beat vectorcardiogram (VCG) that is a function of voltage level;
comparing, over a segment of the average heart beat, the average heart beat VCG with a threshold value; and
indicating whether the average heart beat VCG exceeds or does not exceed the threshold value.

38. A system for determining the presence of atrial fibrillation, comprising:
a computer configured to receive data from at least three orthogonal leads indicative of electrical activity of a heart over multiple heart beats, the computer further configured to:
average the data to obtain an average heart beat;
generate, from the averaged data, an average heart beat vectorcardiogram (VCG) that is a function of voltage level;
compare, over a segment of the average heart beat, the average heart beat VCG with a threshold value; and
identify the average heart beat VCG as either exceeding or not exceeding the threshold value; and
an indicator configured to provide an indication of the identification.

39. A program storage device readable by machine, embodying a program of instructions executable by the machine to perform a method, the method comprising:
obtaining data from at least three orthogonal leads indicative of electrical activity of a heart over multiple heart beats;
averaging the data to obtain an average heart beat;
generating, from the averaged data, an average heart beat vectorcardiogram (VCG) that is a function of voltage level;
comparing, over a segment of the average heart beat, the average heart beat VCG with a threshold value; and
indicating whether average heart beat VCG exceeds or does not exceed the threshold value.
